(19) United States
(12) Patent Application Publication
(10) Pub. No.: US 2024/0242817 A1
(43) Pub. Date: Jul. 18, 2024

(54) VISUALIZATION DEVICE

(71) Applicant: Erbe Elektromedizin GmbH, Tuebingen (DE)

(72) Inventors: Yannick DANIEL, Balingen (DE); Ovidiu JURJUT, Bensheim (DE); Adrian MICHELMANN, Reutlingen (DE)

(21) Appl. No.: 18/411,416

(22) Filed: Jan. 12, 2024

(30) Foreign Application Priority Data
Jan. 17, 2023 (EP) 23152022.2

(51) Int. Cl. G16H 30/40; A61B 1/00; A61B 1/303; A61B 90/00; G06T 7/00; G06T 7/73; G06T 11/00

(52) U.S. Cl. CPC G16H 30/40; A61B 1/00009; A61B 1/303; A61B 90/37; G06T 7/0014; G06T 7/73; G06T 11/00; A61B 2090/365; A61B 2090/372; G06T 2207/10016; G06T 2207/30004; G06T 2210/41

(57) ABSTRACT

A visualization device for diagnosis and therapy monitoring of a treatment of biological tissue is disclosed. The visualization device comprises a camera and an image processor. The latter is configured to detect reference structures present on the tissue in images created by the camera. The image processor detects and tracks optical indicators created by the examination, e.g. in a staining test. The optical indicators can be stained areas. Due to the detection and tracking of the structures, the image processing device determines changes in perspective and creates transformation rules. By means of the latter, the image processing device inserts the indicators, representations and treatment points at the correct position into the reference image irrespective of position changes of the camera, the patient and tissue distortions. The optical indicators characterizing tissue in need of treatment are detected in the correct position independent of patient movements and movements of the treating person.

### CROSS REFERENCE TO RELATED APPLICATION

This application claims the benefit of European Patent Application No. 23152022.2, filed Jan. 17, 2023, which is incorporated herein by reference in its entirety.

### TECHNICAL FIELD

The invention refers to a device for diagnosis and treatment of tissue changes, particularly the diagnosis and treatment of cervical neoplasia. The visualization device according to the invention is also usable in other similar situations.

### BACKGROUND

The treatment of cervix tissue (cervix uteri) can be carried out with video support.

For this purpose the article “Automatic Detection of Anatomical Landmarks in Uterine Cervix Images”, Hayit Greenspan, Shiri Gordon, Gali Zimmerman, Shelly Lotenberg, Jose Jeronimo, Sameer Antani, und Rodney Long, IEEE TRANSACTIONS ON MEDICAL IMAGING, VOL. 28, NO. 3, March 2009, describes the automatic recognition of characteristic points on cervix tissue in order to be able to establish a web-based database about the development of lesions, which are correlated with malign cervical tissue formations.

The feature filtering and image processing is known from “KAZE Features” Pablo F. Alcantarilla, Adrien Bartoli, and Andrew J. Davison Conference Paper October 2012 DOI: 10.1007/978-3-642-33783-3_16 as well as from “Fast Explicit Diffusion for Accelerated Features in Non-Linear Scale Spaces”, Pablo Fernandez Alcantarilla Conference Paper September 2013 DOI:10.5244/C.27.13 and “Speeded-Up Robust Features (SURF)”, Herbert Bay, Andreas Ess, Tinne Tuyetelaars and Luc Van Gool, ETH Zürich BIWI, Sternwartstraβe 7, CH-8092 Zurich, Switzerland preprint submitted to Elsevier of Sep. 10, 2008, compare also https://doi.org/10.1016/j.cviu.2007.09.014.

In addition, a method and a system for cervix position detection by means of ultrasound are known from MY 181157A. For cervix position detection an ultrasound transducer is used, which is linearly movably supported in X-, Y- and Z-direction as well as in addition pivotably supported. An automatic cervix identification algorithm allows a user-independent detection of the cervix in the ultrasound images.

In addition, DE 10 2019 116 381 A1 discloses a method for determination of the image position of a marked point in an image of an image sequence. For this purpose, in a first image a marked point is defined and a transformation between corresponding sub-areas of the first image and a second image of the image sequence is determined. With this transformation at least a sub-area of the first image is transformed. In the transformed image the marked spot is again localized and transferred into the second image by means of the transformation.

In the treatment of cervix tissue it has to be expected during the measures to be taken for diagnosis as well as during the actual treatment measures that the relative position between tissue and camera can be changed. In addition, cervix tissue can carry out movements due to the manipulations that are carried out and also as a result of muscular reactions. As long as a treatment of a tissue shall be uniformly extended over the entire tissue surface, this might be unproblematic. If however an influence only limited to specific areas with need for treatment is desired, it is necessary to identify such areas and localize them over a longer period independent from the camera position or tissue movements.

### SUMMARY

Starting therefrom it is one object of the invention to provide an improved visualization device.

This object is solved by means of the visualization device as described herein.

The visualization device according to the invention serves particularly for diagnosis and therapy monitoring during the treatment of cervix tissue or other biological tissue, particularly tissue that is deformable. The deformation can, for example, result from mechanical influence during surgery or also from a movement of muscular tissue that is to be treated or that is in mechanical connection with the tissue to be treated.

At least one camera for capturing multiple images or a video stream during an examination of tissue is part of the visualization device. The examination can be particularly an examination accompanied by a change of the optical appearance of the tissue, such as an examination by means of a staining test. During the latter the tissue to be examined is brought into contact with a suitable liquid influencing the tissue, e.g. acetic acid solution or Lugol's solution. Subsequently, discolorations are formed on the tissue surface, which characterize tissue features. For example, cervical intraepithelial neoplasia can be made visible in this manner by means of a staining test.

The at least one camera for capturing images of the staining test or during another tissue examination can be, for example, a camera that is stationary set up or also a camera carried by the treating person and thus a movable camera. A movable camera can be, for example, a helmet camera, a camera integrated into VR-glasses of the treating person or another camera carried on the shoulder or head.

In another embodiment the examination can also be carried out solely video-based and/or automated. For example, an optionally provided analysis unit can recognize the treatment area solely on the basis of the camera images or the user can manually define the treatment area in the camera image, e.g. by means of a touch screen or another input device. As optical indicator the coordinates of the defined treatment area can then be used in that this image area is marked as graphical representation in the camera image of the visualization device. This can be carried out, for example, by means of a boundary line that surrounds the area to be treated or also by means of coloring of the area to be treated. Similarly, it is possible that a dosage recommendation in form of a contour map or a false color map is used for marking the area to be treated.

The images or a video sequence provided by the camera are/is transmitted to an image processing device, which is configured to detect and track reference structures present on tissue in the images as well as in addition optical indicators created or gained by the examination.

Reference structures can be anatomical structures and/or structures formed by instruments. Such instruments can be, for example, a speculum or the like, which are visible in the image. It can be instruments by means of which the field of operation is kept open or which are arranged in the operation field. Reference structures can be subject to a temporal change, particularly in case of anatomical structures. For example, tissue can be subject to change by means of mechanical deformation, thermal influence, hemorrhages, swellings or the like.

The image processing device can also be configured to create graphic representations of specific, e.g. optical, indicators. The optical indicators can be tissue areas that have been identified during examination and that can be optically distinguished from surrounding tissue, for example. Such optical indicators can be complemented or replaced by graphic representations. The graphic representations can be lines surrounding the tissue areas, areas covering the tissue areas or the like.

Due to the tracking of the structures, the image processing device can determine the relative position of the camera in relation to the tissue to be examined, independent from changes of perspective that can result from movements of the camera or the patient. The image processing device is in addition configured to insert the optical indicators created by means of the examination in the correct position in each image of a video sequence based on the determined change of perspective. In doing so, the optical indicators or graphic representations thereof can be transferred into a reference image based on the position of the reference structures.

The reference image is a data representation of the examined tissue in a position with relation to the space independent from the position of the patient and the position of the camera. The reference image contains (static or also temporally varying) reference structures identified by means of the image processing device. The image processing device is configured to determine transformations for the images of an image sequence based on the position of the identified reference structures, wherein by means of the transformations, the content of images of the image sequence can be transferred into the reference image in correct position. In doing so, the reference image is independent from movements of the camera or the patient or from tissue distortions; the image processing device calculates the images of an image sequence or a video into the reference image, independent from the perspective.

Each image of the image sequence or the video can contain indicators created by the examination that characterize tissue areas in need of treatment. For example, the indicators are areas that can be distinguished by color. In addition to the indicators or instead of them, graphic representations can be lines, for example, by means of which tissue sections in need of treatment identified in the staining test or in another examination, are enclosed or marked otherwise.

The detected indicators or the graphic representations thereof are related to the reference image. The image processing device is configured to transfer the indicators or the graphic representations thereof into the reference image by means of the transformation rules determined based on the structures. Also, the image processing device can be configured to update the reference structures in the reference image, if they are subject to variation. For this purpose, the image processing device can be configured to detect a change of perspective based on the present reference structures or based on other indications, e.g. by means of a position detection device, and is in addition configured to transfer a variation of the shape or the other appearance (e.g. color or contrast variations of the reference structure) into the reference image.

The image processing device is, in addition, configured to transfer the indicators or graphic representations of areas with need for treatment during the treatment of the tissue from the reference image into the treating person's field of view. The optical indicators or graphic representations thereof are thereby transferred into the treating person's field of view in correct position and indeed in correct position with reference to the respectively recognized reference structures present in the respective live image. For example, the indicators or graphic representations can be displayed in VR-glasses so that they are visible at the correct position in the live image, i.e. in the image perceived by the treating person.

Alternatively, the indicators or graphic representations thereof can be displayed superimposed onto a guide image on a monitor. The live image can be captured during the treatment by means of a stationary or for example a shoulder-supported camera of the treating person. The treating person can set up the monitor in the proximity of the patient in order to monitor its treatment or to check it from time to time.

The visualization device according to the invention does not require direct detection of the camera location, i.e. it does not require a camera position detection system. A perspective adaption is carried out based on the reference structures captured in the images exclusively by means of the images provided by the camera during the diagnosis as well as by the camera during treatment.

The visualization device particularly also allows the treatment and monitoring of the treatment of cervix tissue by means of an influence that does not leave visible traces on the tissue, i.e. during which the optical appearance of the tissue is not changed. Such an influence can be, for example, the influence on the cervix tissue by means of light or non-thermal plasma, e.g. cold plasma, or another energy form or substance, such as medicinal influence, ultrasound influence or the like. The visualization device can be configured to detect the location of influence during the medical treatment and its movement over the tissue and to capture it in form of a trace. The visualization device can be additionally configured to make this trace visible in a monitor representation or another live image, e.g. in VR-glasses. Due to the continuous adaption of the perspective, not only during examination, but also during treatment, it can be guaranteed that the treating person treats the tissue areas in need of treatment exclusively and sufficiently and preserves other tissue areas.

The camera used during diagnosis as well as the camera used during the treatment of tissue can be generally configured differently. It is, however, also possible to use cameras being identical in construction. It is in addition possible to use one and the same camera for the examination and diagnosis as well as for the treatment, e.g. a shoulder-supported camera of the treating person. The camera can also be arranged immovably during the diagnosis, e.g. by means of a support device, and can later be carried or moved by the treating person during the treatment.

The image processing device can be configured to determine transformation rules, e.g. in form of matrices or similar, based on the change of a position of anatomic structures in the images. These transformation rules can characterize changes in the perspective of the images and/or tissue deformations. Changes in the perspective can result, for example, from a change of the camera position in relation to the patient or changes in the position of the patient. The structures used for adaption of the perspective can be particularly the position of a speculum, the margin of the cervix tissue and/or the position of other anatomic structures, such as the cervix channel or its opening surrounding area. Even in case of deformation or distortion of the tissue or displacement or pivot of the camera in relation to the displayed cervix tissue, optical indicators can be coupled into the live image in correct position, which have been recognized during diagnosis.

### BRIEF DESCRIPTION OF THE DRAWINGS

Additional details of advantageous embodiments of the invention are subject of dependent claims as well as the description and the associated drawing. The drawings show:

FIG. 1 the cervix and the image processing device with connected camera during the cervix examination in schematic illustration,

FIG. 2 the image processing device with connected image display device and camera during treatment of the cervix by means of a plasma instrument in schematic illustration.

FIG. 3 functional blocks of the image processing device in schematic illustration,

FIG. 4 the image processing device based on its functional blocks for adaption of perspective during the cervix examination,

FIGS. 5 to 7 the variation of the reference image during the diagnosis,

FIG. 8 the image processing device based on essential functional blocks during treatment of the tissue,

FIG. 9 the image processing device in a modified embodiment based on essential functional blocks during treatment of the tissue.

### DETAILED DESCRIPTION

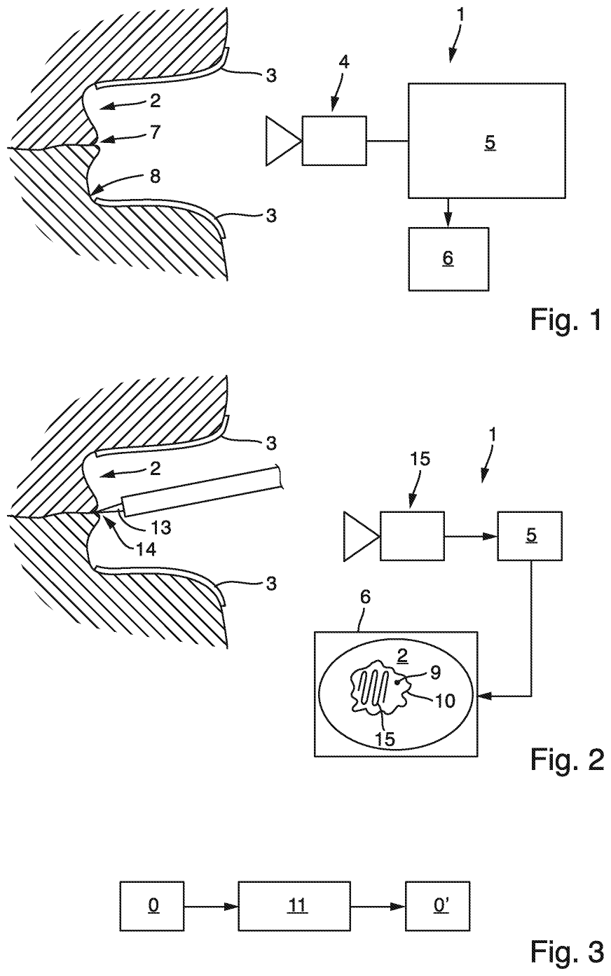

In FIG. 1 a visualization device 1 is illustrated during inspection of biological tissue, e.g. a cervix, which is held by means of a speculum 3 visible for a camera 4. The camera 4 is connected to an image processing device 5. The latter can comprise a monitor or another image display device 6.

The visualization device 1 serves here for examination of cervix tissue, whereby a similar setting can also be used during examination of other tissue 2.

In the present case the tissue examination comprises a staining test during which a suitable essence is applied onto the tissue 2, e.g. acetic acid solution or Lugol's solution. Particularly, intraepithelial neoplasia are discolored in cervix tissue.

However, also staining with fluorescent colorants and their excitation by means of UV/VIS-light or the determination of areas in need of treatment directly from the camera image by means of tissue recognition by using suitable methods is possible.

During the staining test camera 4 captures images or a video sequence and supplies them/it to the image processing device 5. The image processing device 5 thereby serves to detect discolorations occurring in the images of the image sequence and the temporal progress of the discoloration and its abating progress, regardless of potential relative movements between the camera 4 and the tissue 2 and to assign them to the tissue 2 or a model thereof in correct position. During the diagnosis, which can take several minutes, not only the position of the camera 4 in relation to the tissue 2 may change, but also the tissue 2 may distort or deform, e.g. due to muscular actions.

The image processing device 5 is configured to consider both, namely potential deformations of tissue 2, as well as relative movements between tissue 2 and camera 4. The image processing device 5 can be configured to detect anatomic structures present on the tissue 2 in the images, such as the cervix channel 7 or the cervix margin 8, and to use them as orientation points or orientation structures for the adaption of the perspective and the adaption of the distortion of images within the image sequence supplied by camera 4. However, also non-anatomic structures can be used as orientation points or structures, such as the edge of the speculum.

For recognition of the structures two-part methods for feature detection and feature extraction can be used, which are based, for example, on classic segmentation and object recognition based on pixels, edges, regions, clusters, texture, model or shape. Similarly, the recognition of structures can be carried out by means of methods of machine learning, such as semantic segmentation, as well as by means of a combination of different methods.

In addition, the image processing device 5 is configured to detect optical indicators 9 (see FIG. 2) or graphic representations 10 thereof, which have been made visible during the diagnosis (i.e. for example during the staining test) and to display them on the image display device 6, if required (see FIG. 2 for this purpose). For example, such indicators 9 can be areas distinguishable by means of color from remaining tissue 2 created during the staining test. For example, the graphic representations 10 can be edges of the indicators 9 created by means of edge detection routines or similar routines, wherein the edges can be represented in the image of the image display device 6 by means of a polyline.

The diagnosis process is schematically illustrated in FIG. 3. From the images captured at the beginning of the examination a reference image O results. Therein the indicators 9 or graphic representations 10 of subsequently captured images are inserted by means of a transformation block 11, in order to obtain a modified reference image O′ of the examined tissue.

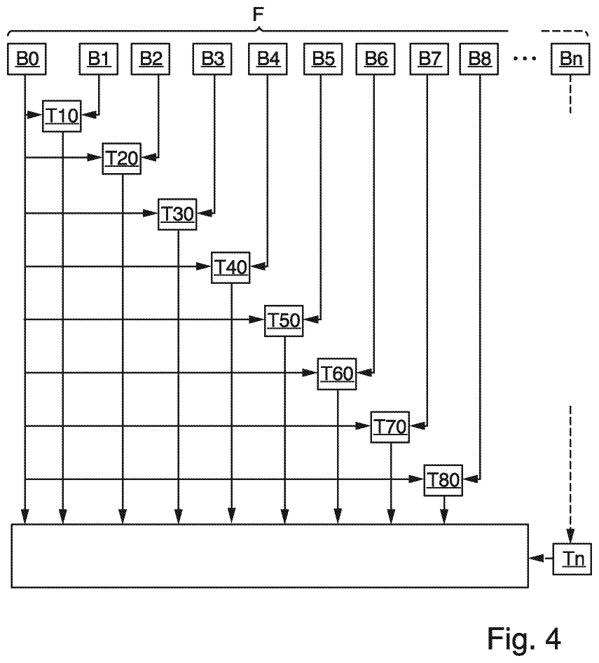

The transformation block 11 is part of the image processing device 5 and serves for transferring indicators 9 in correct position, which are contained in the image sequence supplied by camera 4. FIG. 4 illustrates an image sequence F for this purpose, which can comprise, dependent on the embodiment, a few individual or also hundreds or thousands of images.

In a first image or from the initial image B0 created from the first images and from a subsequent image B1 a transformation T10 is determined. Relative to the image B0 the image B1 can be displaced resulting from a relative movement between tissue 2 and camera 4 or can be distorted due to tilting. In addition, image B1 can contain a distortion that has come about due to a muscle movement of the tissue 2. The image processing device 5 now determines an image displacement as well as an image distortion, particularly from the position of structures that serve as orientation points. For example, the structures can be the margin 8 of tissue 2 or the channel 7 or another characteristic point of tissue 2. A transformation T10 results therefrom. If now color variations of tissue 2 occur during the staining test, they are transferred in correct position into the reference image O′ by means of the transformation T10. Similarly, the process is continued with transformations T20 to T80 as well as transformations for each additional image.

The tracking of anatomic or optical structures can be realized by means of pixel-based methods, such as methods based on phase correlation and frequency range, optical flow and block matching or by means of feature-based methods, such as statistical and filter-based methods. In addition, the tracking can be based on methods of machine learning, such as object tracking or a combination of machine learning and classic tracking. The term “machine learning” can comprise the following and is, however, not limited thereto: artificial neural networks, convolutional neural networks (CNN), recurrent neural networks (RNN), generative adversarial networks (GAN), Bayes Regression, Naive Bayes classifier, nearest neighbors classification, Support Vector Machine (SVM) in addition to other techniques in the field of data analysis.

After termination of the staining test, reference image O′ comprises the indicator 9 and/or the graphic representation 10 in correct position regardless of relative movements between the tissue and camera 4 or the distortion of tissue 2.

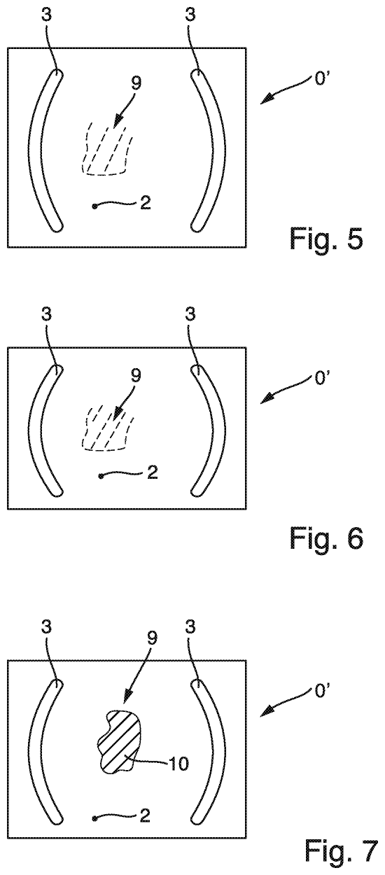

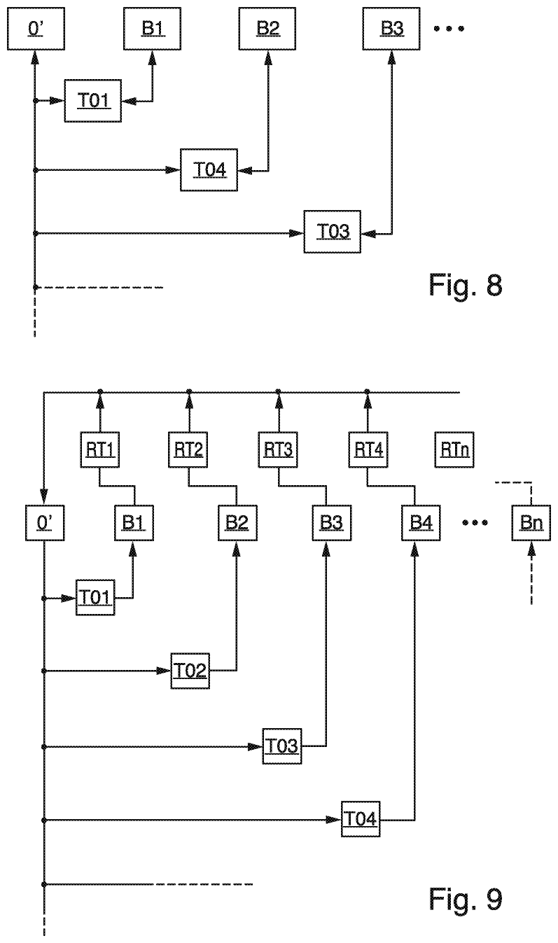

FIGS. 5 to 7 illustrate individual images created during the staining test, which are respectively transferred in correct position into the reference image O′. In FIG. 5 an emerging indicator 9 is apparent that becomes clearer and clearer during the further progress of the staining test, as illustrated by FIGS. 6 and 7. Resulting from the continuously executed transformation of the respective image with regard to its perspective and its distortion into the reference image O, position changes during the staining test do not play any role.

Preferably, the adaption of the perspective described above can be carried out by means of the visualization device 1 not only during the tissue examination, but beyond that also during tissue treatment. For example, if it has been determined during examination that at least one tissue area in need of treatment exists, which is displayed by the indicator 9 or its graphic representation 10, the treating person can chemically or physically influence the tissue 2 in order to achieve a successful therapy. Particularly he or she can thereby carry out an influence that does not leave visible traces on the tissue 2. The visualization device 1 can thereby contribute to limit the treatment on the areas needing treatment limited by indicator 9 or representation 10 and to cause a sufficient treatment there hereafter.

For treatment an instrument 12 is provided to the treating person by means of which tissue 2 has to be locally treated. For example, the instrument 12 can be a laser, a plasma instrument, particularly a cold plasma instrument, an instrument that emits a substance jet or the like.

In the example illustrated in FIG. 2 the instrument 12 creates a jet 13 of cold plasma. The instrument 12 is preferably guided manually. The cold plasma 13 impinges on a point 14 and thus moves with a speed set by the treating person and along a path over the tissue 2 selected by the treating person.

Independent from the type of treatment and the instrument, a camera 15 can be provided for monitoring the treatment, which is connected to the image processing device 5. The camera 15 can be the same camera as camera 4 that has been used during the staining test. It is however also possible to use different cameras, 4, 15 during the examination according to FIG. 1 and during the treatment according to FIG. 2. Particularly camera 15 can be a head-supported or shoulder-supported camera of the treating person.

The image processing device 5 can be configured to reconstruct the point 14 from the image sequence supplied by camera 15 during treatment and the position of the instrument 12 displayed in the images or—in case the plasma lights up sufficiently—to directly determine the point 14 and to insert it into the reference image. In this manner the reference image O′ and the trace 15 of the treatment resulting from the monitoring of the path of point 14 over the tissue 2 can be displayed on the image display device 6.

The image display device 6 can thereby be located in the proximity of the patient, so that the treating person can have a look on the monitor image from time to time and can control his or her treatment there. For image representation the indicator 9 (or its graphic representation 10) and the trace 15 can, however, also be brought into the field of view of the treating person in another manner, e.g. by representation in VR-glasses in which then indicator 9 (or its graphic representation 10) as well as the trace 15 of the treatment are fed in correct position into the real image perceived by the treating person.

For example, camera 15 can be a camera connected to the VR-glasses without separate position determination. The image processing device 5 compares the image B1, B2, B3, etc. captured by the camera with reference image O′ and therefrom determines the transformation T01. With this transformation indicator 9 (or its graphic representation 10) detected during examination is transferred from the reference image O′ into the first image B1.

If in the image B1 a treatment already takes place, the position of the first point 14 of the treatment can be transferred back by means of the transformation T01 into the reference image O′. The same applies for the additional images B2, B3, etc. of the image sequence, so that by means of the retransfer of the points 14 into the reference image O′ the treatment trace 15 is created there.

The transformations T01, T02, T03, etc. thereby achieve a perspective and distortion correction, so that all points 14 and thus the trace 15 are transferred undistorted and in correct position into the reference image O′. By means of the forward transformations T01, T02, T03, etc. it is, however, displayed independent from head movements of the treating person and independent from camera movements in a sense resting in his/her live image in VR-glasses or, as illustrated in FIG. 2, on the monitor or another image display device 6.

In another embodiment illustrated in FIG. 9 it can be provided that the initial image O′ is replaced from time to time by a reference image Oⁿ being more up-to-date and the subsequent transformations T01, T02, . . . are referred back to this reference image Oⁿ. The substitution of the current reference image Oⁿ by means of a new reference image Oⁿ⁺¹ by means of a back transformation RT1, RT2, . . . can either be carried out by manual input of the user or automatically according to a non-variable time interval. Similarly, it is possible that the conformity between the reference image O′ or O″ and the current image Bn is determined and the update of the reference image Oⁿ is carried out in case a defined conformity is not reached. Similarly, it is also possible to monitor the degree of image variation defined by the back transformation RTn as characteristic for a required update of the reference image Oⁿ. It can be provided that the reference image Oⁿ is only updated, if the degree of the image variation exceeds a predefined degree.

The visualization device 1 according to the invention serves particularly for diagnosis and therapy monitoring during treatment of biological tissue 2. It comprises a camera 4 and an image processing device 5 connected thereto. The latter is configured to detect reference structures 7, 8 on the tissue 2 present in the images created by camera 4. It is, in addition, configured to detect and track optical indicators 9 created by the examination, e.g. in a staining test. The optical indicators can be stained areas. Due to the detection and tracking of structures 7, 8, the image processing device 5 determines changes in perspective and creates respective transformation rules. By means of these, the image processing device inserts the indicators 9, representations 10 and treatment points 14 at the correct position in the reference image O, O′, Oⁿ irrespective of position changes of the camera 4, the patient or irrespective of tissue distortions. The optical indicators, which characterize the tissue in need of treatment, are in this manner detected in the correct position and indeed independent from patient movements or movements of the treating person or the camera.

Similarly, the image processing device 5 can detect during the subsequent treatment of tissue 2 the trace 15 of the treatment and take over the trace 15 into the reference image O, O′, Oⁿ. Thereby the image processing device 5 carries out an adaption of the perspective based on reference structures 7, 8 serving as orientation points, e.g. in that it equalizes image distortions caused by a position change of the camera. The image processing device transfers the indicators 9 and/or representations 10 based on the positions of the structures 7, 8 into the reference image O, O′, Oⁿ during the diagnosis. During treatment the image processing device transfers the indicators 9 and/or representations 10 based on the position of the structures 7, 8 into the live image. The visualization device 1 according to the invention thus allows a safe and comfortable treatment without the need of position detection of camera 4, 15 during diagnosis or treatment.

### LIST OF REFERENCE SIGNS

1 visualization device
2 tissue (e.g. cervix uteri)
3 speculum
4 camera
5 image processing device
6 image display device
7 cervix channel
8 cervix margin
9 indicator
10 graphic representation of indicator 9
F image sequence
B0-Bn images of an image sequence F or a video
O original image
O′ modified original image/reference image
Oⁿ reference image after n^{th} actualization
T10-Tn0 transformations for transferring details of actual images into the reference image O, O′ or Oⁿ (during examination)
T01-T0n transformations for transferring details of reference image O, O′ or Oⁿ into the actual image (during treatment)
RT1-RTn transformations for transferring details of actual images into the reference image O, O′ or Oⁿ (during treatment)
11 transformation block
12 instrument
13 cold plasma
14 point where plasma 13 impinges on tissue 2
15 trace

1. A visualization device (1) comprising:
a camera (4) for obtaining a plurality of images (B1, B2 . . . ) during an examination of tissue (2),
an image processing device (5) that is configured to detect and/or track in the plurality of images (B1, B2 . . . ) reference structures present on the tissue (2) as well as optical indicators (9) obtained during the examination, in order to transfer the optical indicators (9) or graphic representations (10) thereof based on a position of the reference structures (7, 8) into a reference image (O, O′, Oⁿ) and transfer the optical indicators (9) or graphic representations (10) thereof during treatment of the tissue from the reference image (O, O′, Oⁿ) into a field of view of a treating person.

2. The visualization device according to claim 1, wherein the camera (4) is configured to be immovably arranged during the obtaining of the plurality of images during the examination of the tissue (2).

3. The visualization device according to claim 1, wherein the camera (4) is configured to be movably supported during the obtaining of the plurality of images during the examination of the tissue (2).

4. The visualization device according to claim 1, further comprising a camera (4, 15) for obtaining a plurality of images (B1, B2 . . . ) during the treatment of the tissue (2).

5. The visualization device according to claim 4, wherein the camera (15) for obtaining a plurality of images (B1, B2 . . . ) during the treatment of the tissue (2) is of a same construction as the camera (4) for obtaining a plurality of images during the examination of the tissue (2).

6. The visualization device according to claim 1, wherein the image processing device (5) is configured to determine transformation rules (T10, T01 . . . ) based on changes of the position of the reference structures (7, 8) in the plurality of images (B1, B2 . . . ) that represent perspective changes of the plurality of images.

7. The visualization device according to claim 1, wherein the image processing device (5) is configured to determine transformation rules (T10, T01 . . . ) based on changes in the position of the reference structures (7, 8) in the plurality of images (B1, B2 . . . ) that characterize tissue deformations.

8. The visualization device according to claim 1, wherein the image processing device (5) is configured to detect changes of the reference structures (7, 8) and to transfer them into the reference image (O, O′, On).

9. The visualization device according to claim 1, wherein the image processing device (5) is configured to determine transformation rules (T10, T01 . . . ) based on changes of the position of the reference structures (7, 8) in the plurality of images (B1, B2 . . . ) that characterize changes in a perspective of the plurality of images (B1, B2 . . . ) and tissue deformations.

10. The visualization device according to claim 1, wherein the image processing device (5) is configured to transfer optical indicators (9) created by the examination of the tissue or graphic representations (10) thereof from the reference image (O, O′, On) into an image optically perceivable by the treating person during the treatment based on the position of the reference structures (7, 8) present on the tissue (2).

11. The visualization device according to claim 1, wherein the image processing device (5) is configured to display optical indicators (9) created by the examination, or their graphic representations (10), from the reference image (O, O′, On) on an image display (6) superimposed with images (B1, B2 . . . ) captured during the treatment.

12. The visualization device according claim 1, wherein the image processing device is configured to output optical indicators (9) obtained by the examination, or their graphic representations (10), from the reference image (O, O′, On) in VR-glasses for superimposition with real images visible by the treating person, wherein the VR-glasses comprise a camera (15) connected to the image processing device (5).

13. The visualization device according to claim 1, wherein the image processing device (5) is configured to record a trace (15) of an influence of an instrument (12) on the tissue (2) during the treatment.

14. The visualization device according to claim 13, wherein the image processing device (5) is configured to transfer the trace (15) recorded during the influence of the instrument (12) on the tissue (2) into the field of view of a treating person.

15. The visualization device according to claim 14, wherein the image processing device (5) is configured to transfer the trace (15) recorded during the influence of the instrument (12) on the tissue (2) into the field of view of a treating person corresponding to a perspective taken by the treating person and/or corresponding to a tissue deformation.
